(19) United States
(12) Patent Application Publication
(10) Pub. No.: US 2023/0371931 A1
(43) Pub. Date: Nov. 23, 2023

(54) ULTRASONIC DIAGNOSTIC APPARATUS AND ULTRASONIC INFORMATION PROCESSING APPARATUS

(71) Applicant: FUJIFILM Healthcare Corporation, Kashiwa-shi (JP)

(72) Inventors: Suguru Ishiguro, Chiba (JP); Koji Waki, Chiba (JP); Eiji Kasahara, Chiba (JP); Akira Kusakabe, Chiba (JP)

(21) Appl. No.: 18/142,083

(22) Filed: May 2, 2023

(30) Foreign Application Priority Data
May 20, 2022 (JP) 2022-082813

(51) Int. Cl. A61B 8/00

(52) U.S. Cl. CPC A61B 8/565; A61B 8/465

(57) ABSTRACT

An ultrasonic diagnostic apparatus is provided with a control unit that transmits to an information processing apparatus ultrasonic diagnostic data generated by transmission and reception of ultrasound, and acquires from the information processing apparatus external processing data in which the ultrasonic diagnostic data have been subjected to processing. The control unit executes a diagnostic apparatus interface program as an interface program common to a plurality of applications installed to the information processing apparatus, and constructs a diagnostic apparatus interface. The diagnostic apparatus interface performs, when the information processing apparatus is caused to execute an execution application that is one of the plurality of the applications, transmission of the ultrasonic diagnostic data and reception of the external processing data with respect to the execution application.

### CROSS REFERENCE TO RELATED APPLICATION

This application claims priority to Japanese Patent Application No. 2022-082813 filed on May 20, 2022, which is incorporated herein by reference in its entirety including the specification, claims, drawings, and abstract.

### TECHNICAL FIELD

The present disclosure relates to an ultrasonic diagnostic apparatus and an ultrasonic information processing apparatus, and specifically relates to a technique of transferring information between the ultrasonic diagnostic apparatus and the ultrasonic information processing apparatus.

### BACKGROUND

Research and development are being conducted on an ultrasonic diagnostic system that connects by communication via an information processing apparatus to an ultrasonic diagnostic apparatus, and causes the information processing apparatus to execute additional image processing. In this system, the ultrasonic diagnostic apparatus displays an image based on image data generated by the information processing apparatus. Processing executed by the information processing apparatus includes processing of extracting data indicating an outline of a tissue from a series of ultrasonic image data (real-time ultrasonic image data) successively generated with the elapse of time by the ultrasonic diagnostic apparatus, and drawing the outline of the tissue on a real-time ultrasonic image, and the like.

Patent Document 1 below describes a technique of transferring medical information from an ultrasonic diagnostic apparatus to an external terminal. In the ultrasonic diagnostic apparatus, transfer conditions when medical information is transferred are set in accordance with a communication environment and the like. The transfer conditions include conditions of transferring medical information without any change, transferring information with high priority, and the like. Patent Document 2 describes a technique of measuring the blood flow rate by the Doppler method, as a technique related to the disclosure.

### CITATION LIST

PATENT DOCUMENT 1: JP 2013-111100 A
PATENT DOCUMENT 2: JP 2015-198777 A

### SUMMARY

In some conventional ultrasonic diagnostic apparatuses, interface software is individually installed to each of a plurality of applications installed to an information processing apparatus. In this case, in a case where an application is additionally installed to the information processing apparatus, an operating condition for the ultrasonic diagnostic apparatus needs to be changed due to a change in a program or the like, so that there has been a problem that work burden for a person who develops the ultrasonic diagnostic apparatus may become large.

An object of the disclosure is to make it easy to additionally install an application to the information processing apparatus, in an ultrasonic diagnostic system in which the information processing apparatus is connected by communication to the ultrasonic diagnostic apparatus.

An ultrasonic diagnostic apparatus according to the disclosure includes a control unit that transmits ultrasonic diagnostic data generated by transmission and reception of ultrasound to an information processing apparatus, and acquires external processing data in which the ultrasonic diagnostic data have been subjected to processing from the information processing apparatus, in which the control unit executes an interface program that is common to a plurality of applications installed to the information processing apparatus, and that performs, when the information processing apparatus is caused to execute an execution application that is one of the plurality of the applications, transmission of the ultrasonic diagnostic data and reception of the external processing data with respect to the execution application.

In one embodiment, by executing the interface program, the control unit refers to an application list that is provided by the information processing apparatus, and in which operating conditions are associated with the respective applications, and transmits to the information processing apparatus, when the information processing apparatus is caused to execute the execution application, information in accordance with the operating condition associated with the execution application.

In one embodiment, the control unit causes, when the information processing apparatus is connected by communication, a display device to display a GUI of each of the applications.

Moreover, an ultrasonic information processing apparatus according to the disclosure includes an information processing unit that acquires ultrasonic diagnostic data generated by an ultrasonic diagnostic apparatus by transmission and reception of ultrasound, in which the information processing unit causes the ultrasonic diagnostic apparatus to refer to an application list in which operating conditions are respectively associated with a plurality of applications with respect to the ultrasonic diagnostic data, executes an execution application that is one of the plurality of the applications in accordance with a command received from the ultrasonic diagnostic apparatus that has referred to the application list, and transmits to the ultrasonic diagnostic apparatus the ultrasonic diagnostic data that have been subjected to processing by the execution application.

In one embodiment, the information processing unit executes an interface program that is common to the plurality of the applications, and each of the applications transmits and receives information to and from the ultrasonic diagnostic apparatus via an interface that is constructed by the interface program.

With the disclosure, it is possible to make it easy to additionally install an application to the information processing apparatus, in an ultrasonic diagnostic system in which the information processing apparatus is connected by communication to the ultrasonic diagnostic apparatus.

### BRIEF DESCRIPTION OF DRAWINGS

Embodiment(s) of the disclosure will be described based on the following figures, wherein:

FIG. 1 is a diagram illustrating an ultrasonic diagnostic system according to an embodiment of the present disclosure;

FIG. 2 is a diagram illustrating configurations of an ultrasonic diagnostic apparatus and an information processing apparatus;

FIG. 3 is a sequence chart when the information processing apparatus executes a j-th application as an execution application; and

FIG. 4 is a diagram illustrating an example of an image to be displayed on a display device.

### DESCRIPTION OF EMBODIMENTS

An embodiment of the disclosure will be described with reference to the respective drawings. The same components illustrated in the plurality of the drawings are assigned the same reference numerals, and repeated descriptions thereof are omitted.

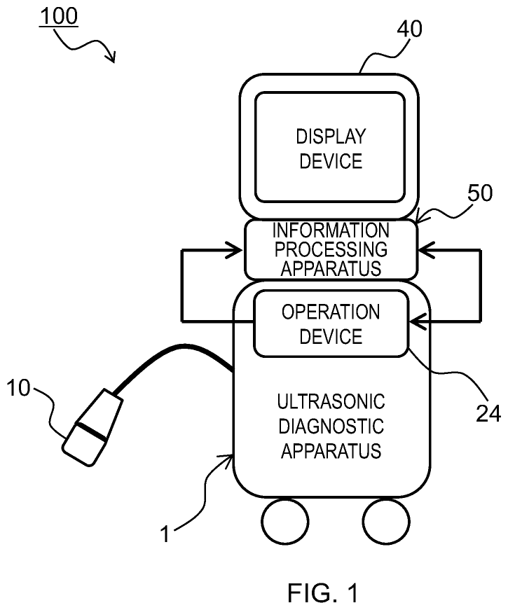

FIG. 1 illustrates an ultrasonic diagnostic system 100 according to an embodiment of the disclosure. The ultrasonic diagnostic system 100 is provided with an ultrasonic diagnostic apparatus 1 and an information processing apparatus 50 (ultrasonic information processing apparatus). The ultrasonic diagnostic apparatus 1 and the information processing apparatus 50 may be connected to each other in a wired manner or in a wireless manner. When a subject is diagnosed, an ultrasonic probe 10 included in the ultrasonic diagnostic apparatus 1 is in a state where a transmission and reception surface at a tip thereof is in contact with a surface of the subject. The ultrasonic diagnostic apparatus 1 causes the ultrasonic probe 10 to transmit ultrasound. The ultrasound transmitted from the ultrasonic probe 10 is reflected in the subject, and is received by the ultrasonic probe 10. The ultrasonic probe 10 converts the received ultrasound into a reception signal that is an electric signal.

The ultrasonic diagnostic apparatus 1 generates image data based on the reception signal. The ultrasonic diagnostic apparatus 1 causes the information processing apparatus 50 to execute additional image processing, and causes a display device 40 of the ultrasonic diagnostic apparatus 1 to display thereon image data generated by the information processing apparatus 50. The ultrasonic diagnostic apparatus 1 may cause the display device 40 to display an image based on image data that are not subjected to the processing by the information processing apparatus 50.

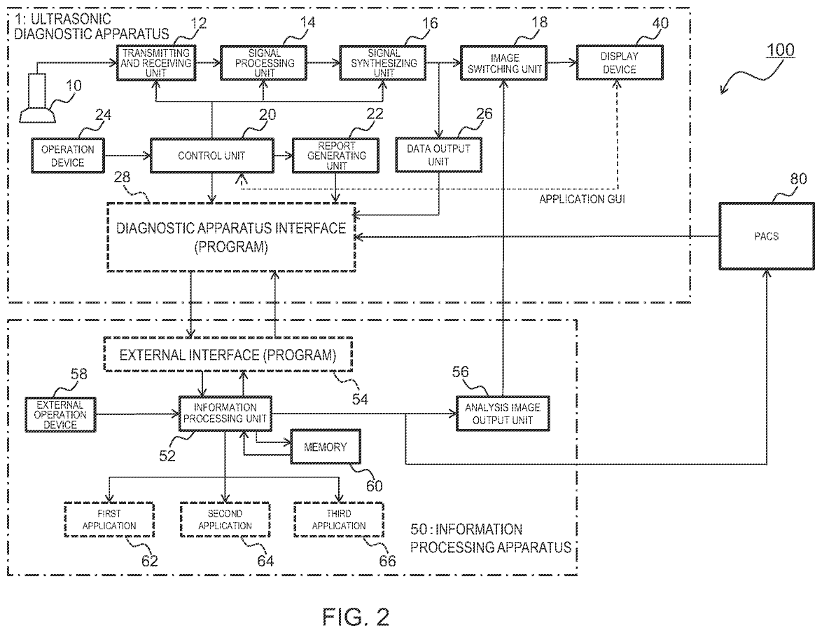

FIG. 2 illustrates a configuration of the ultrasonic diagnostic system 100. The ultrasonic diagnostic system 100 is provided with the ultrasonic diagnostic apparatus 1, the information processing apparatus 50, and a picture archiving and communication system (PACS) 80. The ultrasonic diagnostic apparatus 1 is provided with the ultrasonic probe 10, a transmitting and receiving unit 12, a signal processing unit 14, a signal synthesizing unit 16, an image switching unit 18, the display device 40, a control unit 20, a report generating unit 22, an operation device 24, a data output unit 26, and a diagnostic apparatus interface 28.

The signal processing unit 14, the signal synthesizing unit 16, the image switching unit 18, the control unit 20, the report generating unit 22, and the data output unit 26 may include processors and electronic circuits that execute programs to implement the functions of these components (the signal processing unit 14, the signal synthesizing unit 16, the image switching unit 18, the control unit 20, the report generating unit 22, and the data output unit 26). The image switching unit 18 may include an electronic circuit that implements a communication function. The image switching unit 18 may conduct wired communication or wireless communication.

The control unit 20 executes overall control of the ultrasonic diagnostic apparatus 1. The operation device 24 may be provided with a button, a lever, a keyboard, a mouse, or the like. The operation device 24 may be a touch panel that is provided to the display device 40. The control unit 20 may execute the control of the ultrasonic diagnostic apparatus 1 based on an operation by a user. The control unit 20 transmits and receives information for executing an application by the information processing apparatus 50 to and from the information processing apparatus 50 via the diagnostic apparatus interface 28.

Here, the diagnostic apparatus interface 28 is constructed due to the execution of a diagnostic apparatus interface program by the control unit 20. Further, in FIG. 2, for convenience of explanation, the diagnostic apparatus interface 28 is illustrated by a dashed line independent of the control unit 20. The diagnostic apparatus interface 28 may operate together with an electronic circuit that implements a communication function. The electronic circuit may conduct wired communication or wireless communication.

The ultrasonic probe 10 is provided with a plurality of ultrasonic transducers. The transmitting and receiving unit 12 outputs to the plurality of the ultrasonic transducers transmission signals that are electric signals. Each of the ultrasonic transducers converts the transmission signal into ultrasound, and transmits the ultrasound toward a subject. The transmitting and receiving unit 12 adjusts delay time of the transmission signal to be output to each of the ultrasonic transducers to form an ultrasonic beam in a specific direction.

Each of the ultrasonic transducers receives ultrasound reflected in the subject, converts the ultrasound into a reception signal that is an electric signal, and outputs the reception signal to the signal processing unit 14. The signal processing unit 14 adjusts the delay time of the reception signal output from each of the ultrasonic transducers such that the reception signals that have arrived from the directions toward which the ultrasonic beams have been directed and are based on the ultrasound mutually strengthen, and adds and sums the respective reception signals after adjustment of the delay time. The signal processing unit 14 outputs a phasing addition signal generated in this manner to the signal synthesizing unit 16.

Further, the transmitting and receiving unit 12 changes the delay time of a transmission signal to be output to each of the ultrasonic transducers such that ultrasonic beams are scanned within a specific observation cross-section in the subject. Moreover, the signal processing unit 14 changes the delay time of a reception signal output from each of the ultrasonic transducers so as to generate a phasing addition signal corresponding to the direction of the ultrasonic beam scanned in the subject, and adds and sums the respective reception signals after the delay time adjustment.

The signal synthesizing unit 16 generates B mode image data based on the phasing addition signals acquired in the respective directions within the observation cross-section, and outputs the B mode image data to the image switching unit 18. The transmitting and receiving unit 12, the signal processing unit 14, and the signal synthesizing unit 16 successively generate B mode image data as time elapses, at a predetermined frame rate. Here, the frame rate indicates the number of sheets of B mode images that are generated per unit time.

In basic display processing that is executed in the ultrasonic diagnostic system 100, the image switching unit 18 outputs to the display device 40 the B mode image data successively generated as time elapses. The display device 40 displays an image based on the B mode image data successively generated as time elapses, in other words, a real-time image of a B mode image.

The above description indicates processing of scanning ultrasonic beams in a subject, and generating a B mode image based on phasing addition signals corresponding to the ultrasonic beams in the respective directions. The ultrasonic diagnostic apparatus 1 may execute an operation in a Doppler mode in which a rate of the blood flow is obtained based on a difference (Doppler shift) in a frequency of the phasing addition signal with respect to a frequency of the transmission signal. The control unit 20 generates, for example, Doppler data indicating the rate of the blood flow in a predetermined range set on the ultrasonic beams in the respective directions.

The control unit 20 outputs to the report generating unit 22 ultrasonic diagnostic data such as B mode image data and Doppler data acquired with respect to the ultrasonic beams in the respective directions. The report generating unit 22 generates an ultrasonic test report based on the ultrasonic diagnostic data. The ultrasonic test report is data indicating information including the size and shape of a tissue, whether findings are noticed, and the like. The ultrasonic test report may be generated in an expression form (format) in accordance with a tissue to be diagnosed.

The ultrasonic diagnostic system 100 may execute application display processing below. The signal synthesizing unit 16 outputs to the data output unit 26 the ultrasonic diagnostic data successively generated as time elapses. The data output unit 26 outputs to the diagnostic apparatus interface 28 the ultrasonic diagnostic data successively generated as time elapses. The diagnostic apparatus interface 28 transmits the ultrasonic diagnostic data to the information processing apparatus 50.

Applications that subject the ultrasonic diagnostic data to specific processing are installed to the information processing apparatus 50. The applications include a first application 62 that generates streaming image data based on the B mode image data successively generated as time elapses. The streaming image data indicate data for displaying a real-time image based on the B mode image data successively generated as time elapses.

The information processing apparatus 50 executes the application to generate application analysis data (hereinafter, referred to as APP analysis data) as external processing data. The APP analysis data may be data indicating a numerical value, or may be image data. In a case where the APP analysis data are image data, an information processing unit 52 outputs the APP analysis data to an analysis image output unit 56. The analysis image output unit 56 transmits the APP analysis data to the image switching unit 18, and the image switching unit 18 outputs the APP analysis data to the display device 40. The display device 40 displays an image based on the APP analysis data.

The configuration of the information processing apparatus 50 and the processing to be executed by the information processing apparatus 50 will be specifically described. The information processing apparatus 50 is provided with the information processing unit 52, an external interface 54, the analysis image output unit 56, an external operation device 58, a memory 60, the first application 62, a second application 64, and a third application 66. The information processing apparatus 50 may include processors and electronic circuits that execute programs to implement functions of the respective components (the information processing unit 52, the external interface 54, the analysis image output unit 56, the first application 62, the second application 64, and the third application 66). The analysis image output unit 56 may include an electronic circuit that implements a communication function. The analysis image output unit 56 may conduct wired communication or wireless communication.

The information processing apparatus 50 may be a general computer such as a personal computer, a tablet computer, or a workstation. The information processing unit 52 performs overall control of the information processing apparatus 50. The external operation device 58 may be a keyboard, a mouse, or the like. In a case where the information processing apparatus 50 is provided with a display device such as a display, the external operation device 58 may be a touch panel included in the display device. The external operation device 58 may be configured integrally with the operation device 24 of the ultrasonic diagnostic apparatus 1 by common hardware.

The information processing unit 52 may control the information processing apparatus 50 in accordance with an operation of the external operation device 58 by the user. The external interface 54 transmits and receives signals to and from the diagnostic apparatus interface 28. Further, in FIG. 2, for convenience of explanation, the external interface 54 is illustrated by a dashed line independent of the information processing unit 52.

Here, the external interface 54 is a component that is constructed due to the execution of an external interface program by the information processing unit 52. The external interface 54 may operate together with an electronic circuit that implements a communication function. The electronic circuit may conduct wired communication or wireless communication.

The first application 62 to the third application 66 are respectively components that are constructed due to the execution of a first application program to a third application program by the information processing unit 52. Further, in FIG. 2, for convenience of explanation, the first application 62 to the third application 66 are illustrated by dashed lines independent of the information processing unit 52.

The first application 62 to the third application 66 transmit and receive information different from one another to and from the ultrasonic diagnostic apparatus 1. Meanwhile, the external interface 54 and the diagnostic apparatus interface 28 are interfaces common to the first application 62 to the third application 66, and transmit and receive information in response to operations of the respective applications. In other words, each of the first application 62 to the third application 66 includes no individual interface for transmitting and receiving information to and from the ultrasonic diagnostic apparatus 1, and transmits and receives information to and from the ultrasonic diagnostic apparatus 1 via the external interface 54 and the diagnostic apparatus interface 28 that are constructed common to the first application 62 to the third application 66.

Before the ultrasonic diagnostic apparatus 1 executes processing of generating ultrasonic diagnostic data, the information processing unit 52 transmits an application list to the control unit 20 via the external interface 54 and the diagnostic apparatus interface 28. The application list may be information in which operating conditions such as an activation command, data designation information, and an operational parameter are associated with the information for identifying each of the applications installed to the information processing apparatus 50. The control unit 20 may cause the display device 40 to display a graphical use interface (GUI) with respect to each of the applications based on the application list.

The application to be installed to the information processing apparatus 50 can be added or removed. In a case where the application is added or removed, the application list is updated in accordance with the addition or the removal. The information processing unit 52 may execute an update of the application list when the information processing unit 52 executes the processing of adding or removing the application.

Here, the activation command is a command for activating the application. The data designation information is information for designating data that are used in the application to be activated. For example, in the first application 62 that displays a streaming image, the data designation information designates B mode image data. Examples of the operational parameters include the depth of an observation range, a gain with respect to the reception signal, time gain control (control value for adjusting a state where the gain is increased as the reception time elapses), and a focus (the degree of converging the ultrasonic beams). The operational parameter may further include a control value for adjusting the image quality.

The first application 62 in the embodiment is an application (streaming application) to display a streaming image. The second application 64 is an application (color Doppler application) to display a color Doppler image. In the color Doppler application, ultrasonic diagnostic data, such as B mode image data and Doppler data, transmitted from the ultrasonic diagnostic apparatus 1 to the information processing apparatus 50 over a predetermined number of frames are stored in the memory 60.

The information processing unit 52 that executes the color Doppler application may execute processing below as described in Patent Document 2, for example. The information processing unit 52 extracts outline data indicating an outline (pattern) of a heart from B mode image data for each frame. The information processing unit 52 obtains a rate vector of the blood flow at each position in the heart from the Doppler data and the outline data in the predetermined number of frames, and generates Doppler image data indicating the rate vector of the blood flow at each position in the heart. The Doppler image data may be data indicating the magnitude of the rate vector as the luminance, and indicating the direction of the rate vector as the hue. The Doppler image data may be, for example, image data in which the rate vector in a direction separating from the ultrasonic probe 10 is indicated in blue pixels, and the rate vector in a direction approaching the ultrasonic probe 10 is indicated in red pixels. The Doppler image data may be image data indicating the rate vector at each observation position as a figure such as an arrow.

The third application 66 in the embodiment is an application (AI analysis application) to perform an AI analysis. In the AI analysis application, APP analysis data (another APP analysis data) obtained by another application, an ultrasonic test report, and the like are stored in the memory 60. The data to be stored in the memory 60 may be transmitted from the ultrasonic diagnostic apparatus 1 to the information processing apparatus 50. In the memory 60, data (machine learning model data) indicating a machine learning model necessary for the AI analysis are stored in advance by machine learning. The AI analysis application determines whether a lesion occurs in the subject based on another APP analysis data, the ultrasonic test report, the machine learning model data, and the like.

In a case where APP analysis data generated by execution of the application are image data, the information processing unit 52 outputs the APP analysis data to the analysis image output unit 56. The analysis image output unit 56 transmits the APP analysis data to the image switching unit 18. The image switching unit 18 outputs the APP analysis data to the display device 40, and the display device 40 displays an image based on the APP analysis data.

The information processing unit 52 may output to the analysis image output unit 56 APP analysis data that are successively generated as time elapses as real-time image data. In this case, the analysis image output unit 56 successively transmits the APP analysis data to the image switching unit 18 as time elapses, and the image switching unit 18 successively transmits the APP analysis data to the display device 40 as time elapses. The display device successively displays an image based on the APP analysis data output from the image switching unit 18 as a real-time image, as time elapses.

The information processing unit 52 may transmit the APP analysis data generated by the execution of the application to the PACS 80, which is a database. The PACS 80 stores the APP analysis data therein. The control unit 20 reads in the APP analysis data from the PACS 80 via the diagnostic apparatus interface 28. The control unit 20 may execute processing of causing the display device 40 to display information indicated by the APP analysis data.

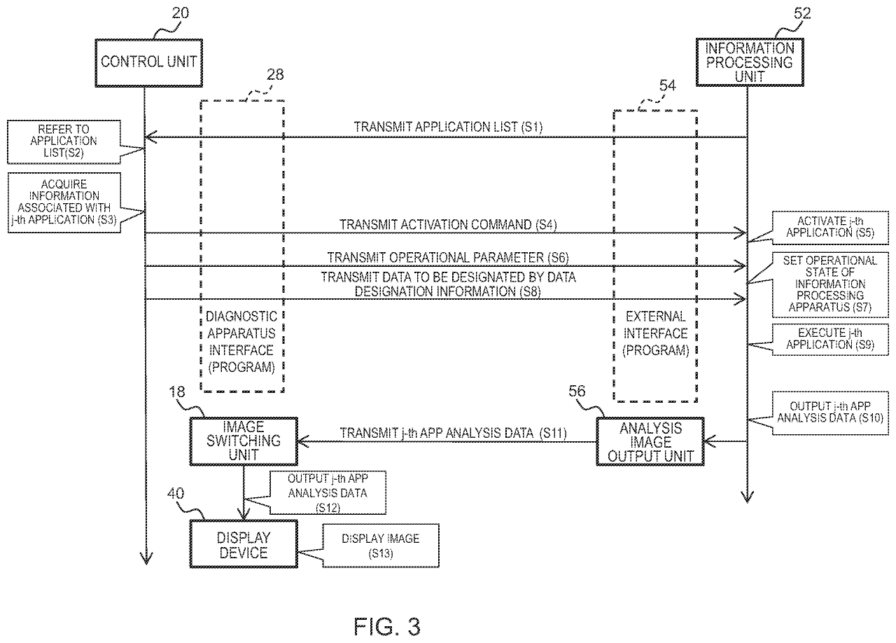

FIG. 3 illustrates a sequence chart when the information processing apparatus 50 executes a j-th application as an execution application. Further, j is an integer of 1 to 3. Moreover, in the example illustrated in FIG. 3, j-th APP analysis data obtained by the j-th application are image data. Before the execution of each application, the information processing unit 52 transmits an application list to the control unit 20 via the external interface 54 and the diagnostic apparatus interface 28 (S1).

When the operation device 24 performs an operation for executing the j-th application, the control unit 20 refers to the application list (S2). The control unit 20 acquires information associated with the j-th application in the application list (S3). In other words, the control unit 20 acquires an activation command, data designation information, and an operational parameter associated with the j-th application (S3). The control unit 20 transmits the activation command for the j-th application to the information processing unit 52 via the diagnostic apparatus interface 28 and the external interface 54 (S4).

When the information processing unit 52 acquires the activation command for the j-th application, the information processing unit 52 activates the j-th application (S5). The control unit 20 transmits the operational parameter corresponding to the j-th application to the information processing unit 52 via the diagnostic apparatus interface 28 and the external interface 54 (S6). The information processing unit 52 sets an operational state of the information processing apparatus 50 in accordance with the operational parameter (S7). In addition, the control unit 20 transmits data designated by the data designation information to the information processing unit 52 via the diagnostic apparatus interface 28 and the external interface 54 (S8).

The information processing unit 52 executes the processing of the j-th application to the data to be designated by the data designation information (S9). The information processing unit 52 outputs to the analysis image output unit 56 j-th APP analysis data obtained by execution of the j-th application (S10). The analysis image output unit 56 transmits the j-th APP analysis data to the image switching unit 18 (S11). The image switching unit 18 outputs the j-th APP analysis data to the display device 40 (S12). The display device 40 displays an image based on the j-th APP analysis data (S13).

In a case of j=1, in other words, in a case where the application is the first application 62, in the application list, data designation information associated with the first application 62 designates B mode image data. The information processing unit 52 executes the first application 62 to successively output to the analysis image output unit 56, as data indicating a real-time image, B mode image data (first APP analysis data) as time elapses (S10). The analysis image output unit 56 successively transmits to the image switching unit 18 the B mode image data as time elapses (S11). The image switching unit 18 outputs the B mode image data to the display device 40 (S12). The display device 40 displays an image based on the B mode image data (S13). Therefore, the B mode image as a real-time image is displayed on the display device 40.

In a case of j=2, in other words, in a case where the application is the second application 64, in the application list, data designation information associated with the second application 64 designates B mode image data and Doppler data. The information processing unit 52 executes the second application 64 to generate color Doppler image data over a plurality of frames as second APP analysis data over a plurality of frames, and to store the color Doppler image data in the memory 60. The information processing unit 52 reads out the color Doppler image data (second APP analysis data) in chronological order from the memory 60, and outputs the color Doppler image data to the analysis image output unit 56 (S10). The analysis image output unit 56 transmits the color Doppler image data to the image switching unit 18 in chronological order (S11). The image switching unit 18 outputs the color Doppler image data to the display device 40 (S12). The display device 40 displays an image based on the color Doppler image data (S13). Therefore, color Doppler images in chronological order are successively displayed on the display device 40.

In a case of j=3, in other words, in a case where the application is the third application 66, in the application list, data designation information associated with the third application 66 designates second APP analysis data (another APP analysis data) obtained by execution of the second application 64, and the ultrasonic test report. The information processing unit 52 executes the third application 66 to determine whether a lesion occurs in the subject. The information processing unit 52 outputs to the analysis image output unit 56 third APP analysis data indicating a determination result (S10). The analysis image output unit 56 transmits the third APP analysis data to the image switching unit 18 (S11). The image switching unit 18 outputs the third APP analysis data to the display device 40 (S12). The display device 40 displays an image based on the third APP analysis data (S13).

Accordingly, the ultrasonic diagnostic apparatus 1 according to the embodiment of the disclosure is provided with the control unit 20 that transmits to the information processing apparatus 50 ultrasonic diagnostic data generated by transmission and reception of ultrasound, and acquires external processing data in which the ultrasonic diagnostic data have been subjected to processing from the information processing apparatus 50. The control unit 20 executes a diagnostic apparatus interface program as an interface program common to a plurality of applications installed to the information processing apparatus 50, and constructs the diagnostic apparatus interface 28. The diagnostic apparatus interface 28 performs, when the information processing apparatus 50 is caused to execute an execution application that is one of the plurality of the applications, transmission of the ultrasonic diagnostic data and reception of the external processing data with respect to the execution application.

The control unit 20 executes the diagnostic apparatus interface program to refer to the application list that is provided by the information processing apparatus. The application list is a list in which operating conditions are associated with the respective applications. The control unit 20 transmits to the information processing apparatus 50, when the information processing apparatus 50 is caused to execute the execution application, information in accordance with the operating condition associated with the execution application.

Moreover, the information processing apparatus 50 (ultrasonic information processing apparatus) according to the embodiment of the disclosure is provided with the information processing unit 52 that acquires ultrasonic diagnostic data generated by the ultrasonic diagnostic apparatus 1 due to transmission and reception of ultrasound. The information processing unit 52 causes the ultrasonic diagnostic apparatus 1 to refer to an application list in which operating conditions are respectively associated with a plurality of applications with respect to the ultrasonic diagnostic data. The information processing unit 52 executes an execution application that is one of the plurality of the applications in accordance with a command received from the ultrasonic diagnostic apparatus 1 that has referred to the application list, and transmits to the ultrasonic diagnostic apparatus 1 ultrasonic diagnostic data that have been subjected to the processing by the execution application.

The information processing unit 52 executes an external interface program that is common to the plurality of the applications, and each of the applications transmits and receives information to and from the ultrasonic diagnostic apparatus 1 via an external interface that is constructed by the external interface program.

In the ultrasonic diagnostic system 100 according to the embodiment, as an interface common to a plurality of applications, the diagnostic apparatus interface 28 and the external interface 54 are respectively constructed in the ultrasonic diagnostic apparatus 1 and the information processing apparatus 50. No interface for transmitting and receiving information to and from the ultrasonic diagnostic apparatus 1 is individually provided to each of the plurality of the applications. The plurality of the applications transmit and receive information to and from the ultrasonic diagnostic apparatus 1 via the external interface 54 and the diagnostic apparatus interface 28 constructed common to the plurality of the applications.

Accordingly, an application compatible to the external interface 54 and the diagnostic apparatus interface 28 is configured, so that the application can be additionally installed to the information processing apparatus 50. Therefore, it becomes easy to additionally install the application to the information processing apparatus 50.

Further, a fourth application that executes processing below may be additionally installed to the information processing apparatus 50. In the application list, data designation information to be associated with the fourth application designates a B mode image.

The information processing unit 52 that executes the fourth application receives B mode image data successively transmitted as time elapses from the control unit 20 via the diagnostic apparatus interface 28 and the external interface 54. The B mode image data successively received as time elapses indicate an ultrasonic image in real time (hereinafter, may be referred to as a real-time ultrasonic image). The information processing unit 52 executes processing of extracting an outline of a tissue from the real-time ultrasonic image, and drawing the outline of the tissue on the real-time ultrasonic image.

The information processing unit 52 generates outline data indicating the outline of the tissue from B mode image data successively transmitted as the time elapses, synthesizes the outline data and the B mode image data, and successively generates outline-B mode image data as fourth APP image data, as time elapses. The outline-B mode image data are data indicating an image in which a figure indicating the outline (figure of the outline drawn by the line) is overlapped with the B mode image.

Moreover, a fifth application that executes processing below may be additionally installed to the information processing apparatus 50. In the application list, data designation information to be associated with the fifth application designates a B mode image.

The image processing to be executed in the fifth application may be processing (fusion display processing) of arranging side by side a basic image such as a magnetic resonance imaging (MRI) image or a computed tomography (CT) image, and a real-time ultrasonic image, relative to the common observation cross-section in the subject. The information processing unit 52 reads in the basic image stored in advance in the memory 60. Moreover, the information processing unit 52 may read in a basic image from an external apparatus. In order to execute the fusion display processing, for example, a position sensor is provided to the ultrasonic probe 10. Based on a position of the ultrasonic probe 10 detected by the position sensor, a basic image in a cross-section where a real-time ultrasonic image has been observed is displayed side by side with the real-time ultrasonic image.

The information processing unit 52 synthesizes an image indicating basic image data with B mode image data, and successively generates basic-B mode image data indicating the basic image and the B mode image by being arranged side by side as fifth APP image data, as time elapses.

In the ultrasonic diagnostic system 100 according to the embodiment, when the ultrasonic diagnostic apparatus 1 and the information processing apparatus 50 are connected to each other so as to be communicable, the control unit 20 may generate image data indicating an application graphical user interface (GUI), and cause the display device 40 to display the image data. The application GUI has a function of the GUI with respect to the application installed to the information processing apparatus 50.

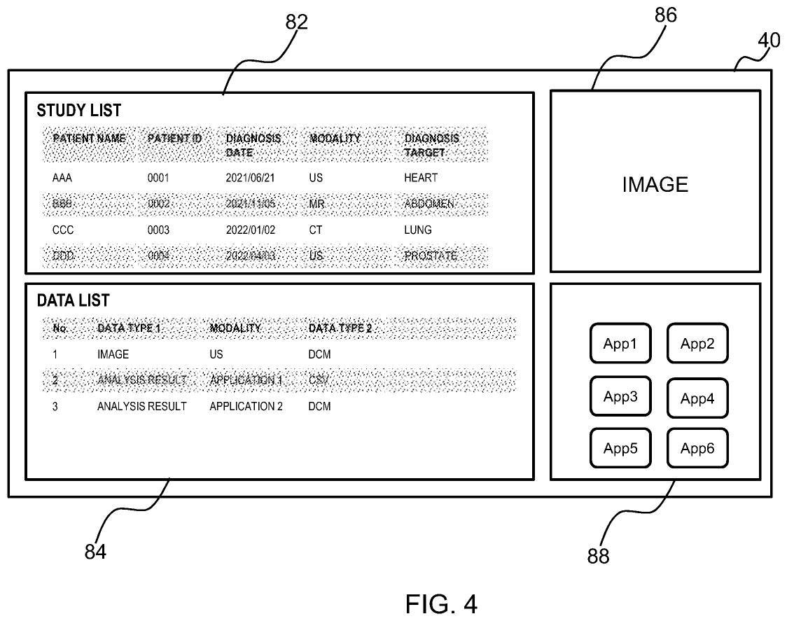

FIG. 4 illustrates an example of an image to be displayed on the display device 40. A study list 82 is displayed in the upper-left part of a display screen, and a data list 84 is displayed in the lower-left part of the display screen. The study list 82 and the data list 84 are lists of data indicating diagnosis results stored in the ultrasonic diagnostic apparatus 1 or the PACS 80. In the study list 82, a patient ID, a diagnosis date, a modality, and a diagnosis target are associated with a patient name. “US” in the modality indicates that data specified by the patient name or the patient ID are data obtained by the ultrasonic diagnostic apparatus. “MR” indicates that data specified by the patient name or the patient ID are data obtained by magnetic resonance imaging (MRI). “CT” indicates that data specified by the patient name or the patient ID are data obtained by computed tomography (CT).

In the data list 84, a data type 1, a modality, and a data type 2 are associated with a data number (No.). “IMAGE” in the data type 1 indicates that data identified by the data number are image data. “ANALYSIS RESULT” indicates that data identified by the data number are expressed by a numerical value. “US” in the modality indicates that data identified by the data number are data obtained by the ultrasonic diagnostic apparatus. “APPLICATION 1” in the modality indicates that data identified by the data number are data obtained by the first application 62. “APPLICATION 2” in the modality indicates that data identified by the data number are data obtained by the second application 64. “DCM” in the data type 2 indicates that a format of data identified by the data number conforms to the standard of digital imaging and communications in medicine (DICOM). “CSV” in the data type 2 indicates that a format of data identified by the data number is comma separated value (CSV).

As for the study list 82, for example, the display column of a patient name or a patient ID is clicked by a cursor to display on the display device 40 an image based on data corresponding to the patient name or the patient ID. As for the data list 84, for example, the display column of the data number is clicked by the cursor to display an image based on data corresponding to the data number on the display device 40.

An image 86 is displayed in the upper-right part of the display screen. Moreover, an application GUI 88 is displayed in the lower-right part thereof. FIG. 4 illustrates an example in which a first application to a sixth application are installed to the information processing apparatus 50. In the application GUI 88, a button App 1 to a button App 6 are respectively provided to the first application to the sixth application. When one of the button App 1 to the button App 6 is clicked, the application corresponding to the clicked button is activated.

In this manner, the application GUI 88 is displayed to show a user the applications that can be executed by the information processing apparatus 50. Therefore, the user easily recognizes what kind of application is executable, and an operation to execute the application becomes easy.

Note that, in the above, as illustrated in FIG. 1, the embodiment in which the information processing apparatus 50 is disposed near the ultrasonic diagnostic apparatus 1 has been indicated. The information processing apparatus 50 may be disposed at a position separate from the ultrasonic diagnostic apparatus 1. For example, the information processing apparatus 50 may be disposed in a room different from a room where the ultrasonic diagnostic apparatus 1 has been disposed. The information processing apparatus 50 may be connected to the ultrasonic diagnostic apparatus 1 via a communication channel such as a local area network.

1. An ultrasonic diagnostic apparatus comprising a control unit that transmits to an information processing apparatus ultrasonic diagnostic data generated by transmission and reception of ultrasound, and acquires from the information processing apparatus external processing data in which the ultrasonic diagnostic data have been subjected to processing, wherein
the control unit executes an interface program that is common to a plurality of applications installed to the information processing apparatus, and that performs, when the information processing apparatus is caused to execute an execution application that is one of the plurality of the applications, transmission of the ultrasonic diagnostic data and reception of the external processing data with respect to the execution application.

2. The ultrasonic diagnostic apparatus according to claim 1, wherein
by executing the interface program, the control unit
refers to an application list that is provided by the information processing apparatus, and in which operating conditions are associated with the respective applications, and
transmits to the information processing apparatus, when the information processing apparatus is caused to execute the execution application, information in accordance with the operating condition associated with the execution application.

3. The ultrasonic diagnostic apparatus according to claim 1, wherein the control unit causes, when the information processing apparatus is connected by communication, a display device to display a GUI of each of the applications.

4. The ultrasonic diagnostic apparatus according to claim 2, wherein the control unit causes, when the information processing apparatus is connected by communication, the display device to display the GUI of each of the applications.

5. An ultrasonic information processing apparatus comprising an information processing unit that acquires ultrasonic diagnostic data generated by an ultrasonic diagnostic apparatus by transmission and reception of ultrasound, wherein
the information processing unit
causes the ultrasonic diagnostic apparatus to refer to an application list in which operating conditions are respectively associated with a plurality of applications with respect to the ultrasonic diagnostic data,
executes an execution application that is one of the plurality of the applications in accordance with a command received from the ultrasonic diagnostic apparatus that has referred to the application list, and
transmits to the ultrasonic diagnostic apparatus the ultrasonic diagnostic data that have been subjected to processing by the execution application.

6. The ultrasonic information processing apparatus according to claim 5, wherein the information processing unit executes an interface program that is common to the plurality of the applications, and each of the applications transmits and receives information to and from the ultrasonic diagnostic apparatus via an interface that is constructed by the interface program.
